(19) United States
(12) Patent Application Publication
(10) Pub. No.: US 2026/0188483 A1
(43) Pub. Date: Jul. 2, 2026

(54) VIRTUAL ASSISTANT FOR INTERCONNECTING MEDICAL DEVICES

(71) Applicant: CareFusion 303, Inc., San Diego, CA (US)

(72) Inventors: Christopher DiLeo, San Diego, CA (US); Brendan Burgess, San Diego, CA (US); Lisa Diggett, San Diego, CA (US); Michael Workman, San Diego, CA (US)

(21) Appl. No.: 18/995,013

(22) PCT Filed: Jul. 21, 2022
(86) PCT No.: PCT/US2022/037935

§ 371 Date: Jan. 15, 2025

(51) Int. Cl. G16H 40/67; G06F 21/34; G16H 10/65; G16H 40/20

(52) U.S. Cl. CPC G16H 40/67; G06F 21/34; G16H 10/65; G16H 40/20

(57) ABSTRACT

A method may include receiving a selection of an order associated with a patient. A scannable token, such as a barcode, may be generated for interacting with one or more medical devices involved in a patient encounter to fulfill the order. In some cases, the same scannable token may be further associated with one or more additional patient encounters associated with the same patient or different patients and conducted by a same clinician or different clinicians. The scannable token may be sent to a client device. In response to the scannable token being scanned at the one or more medical devices, at least a portion of information associated with the order may be sent to the one or more medical devices. Related methods and articles of manufacture are also disclosed.

### TECHNICAL FIELD

The subject matter described herein relates generally to device integration and more specifically to a virtual assistant for providing a software-based interconnection between various medical devices.

### BACKGROUND

A clinician may interact with a variety of medical devices when administering care to various patients. For example, the clinician may dispense, from a dispensing cabinet, one or more medications for the patient. In cases where the one or more medications are administered intravenously, the clinician may configure an infusion pump to deliver the one or more medications. Where the one or more medications include controlled and/or hazardous substances, the clinician may interact with a wasting station in order to dispose any unused portions of the one or more medications.

Clinicians typically have limited time. A clinician's time is typically spent with device interactions, interacting with patients, conferring with clinical colleagues, and other tasks. As devices increase in sophisticated, the time spent interacting and configuring the device may also increase. Time spent at any one device is time that cannot be spent with patients.

### SUMMARY

Systems, methods, and articles of manufacture, including computer program products, are provided for a virtual assistant that interconnects different medical devices in order to expedite interactions with the medical devices thereby allowing more time for an encounter with a patient. In some example embodiments, upon receiving information for the encounter with the patient, an assistant server may generate a token for interacting with one or more medical devices involved in the encounter. Scanning the token at a medical device involved in the encounter may trigger one or more actions associated with the encounter including, for example, configuration of the medical device, generating one or more records of the actions, and/or the like. In instances where the token is associated with a limited lifespan, scanning the token at a medical device after the expiration of the token may fail to trigger any action.

In one aspect, there is provided a system for conducting a scannable token based patient encounter. The system may include at least one data processor and at least one memory. The at least one memory may store instructions that result in operations when executed by the at least one data processor. The operations may include: receiving a selection of a first order associated with a first patient; generating a scannable token for interacting with one or more medical devices involved in a first patient encounter to fulfill the first order; sending, to a client device, the scannable token; and in response to the scannable token being scanned at the one or more medical devices, sending, to the one or more medical devices, at least a portion of information associated with the first order.

In another aspect, there is provided a method for conducting a scannable token based patient encounter. The method may include: receiving a selection of a first order associated with a first patient; generating a scannable token for interacting with one or more medical devices involved in a first patient encounter to fulfill the first order; sending, to a client device, the scannable token; and in response to the scannable token being scanned at the one or more medical devices, sending, to the one or more medical devices, at least a portion of information associated with the first order.

In another aspect, there is provided a non-transitory computer readable medium storing instructions that result in operations when executed by at least one data processor. The operations may include: receiving a selection of a first order associated with a first patient; generating a scannable token for interacting with one or more medical devices involved in a first patient encounter to fulfill the first order; sending, to a client device, the scannable token; and in response to the scannable token being scanned at the one or more medical devices, sending, to the one or more medical devices, at least a portion of information associated with the first order.

In some variations of the methods, systems, and non-transitory computer readable media, one or more of the following features can optionally be included in any feasible combination. The scannable token may be associated with a limited lifespan. The scannable token may expire upon the scannable token reaching an end of the limited lifespan without being scanned at the one or more medical devices.

In some variations, the limited lifespan of the scannable token may be extended in response to the scannable token being scanned a threshold quantity of times and/or at a threshold quantity of the one or more medical devices prior to the expiration of the scannable token.

In some variations, the scannable token may expire prior to an end of the limited lifespan of the scannable token in response to determining that the first patient encounter is complete without the scannable token being scanned at any of the one or more medical devices.

In some variations, the operations may further include: verifying the scannable token scanned at the one or more medical devices prior to sending, to the one or more medical devices, at least the portion of the information associated with the first order.

In some variations, the operations may further include: authenticating, based at least on the scannable token scanned at the one or more medical devices, an identity of a clinician interacting with the one or more medical devices.

In some variations, the scannable token may be an additional authentication factor for authenticating the identity of the clinician interacting with the one or more medical devices.

In some variations, at least the portion of the information sent to the one or more medical devices may include one or more parameters to configure the one or more medical devices for the first order.

In some variations, the one or more medical devices may include an infusion pump. At least the portion of the information sent to the one or more medical devices may include one or more infusion parameters associated the first patient and/or a medication being delivered to the first patient.

In some variations, the one or more data medical devices may include a dispensing cabinet. At least the portion of the information sent to the one or more medical devices may unlock a first receptacle from which to retrieve a medication included in the first order and/or a second receptacle for returning an unused portion of the medication after the medication is administered to the first patient.

In some variations, at least the portion of the information sent to the one or more medical devices may trigger a generation of one or more electronic records documenting an interaction with the one or more medical devices to fulfill the first order.

In some variations, the operations may further include: receiving a patient encounter command specifying the first patient; in response to the patient encounter command, retrieving, from an electronic medical record (EMR) repository, one or more orders associated with the first patient; sending, to the client device, the one or more orders for output at the client device; and receiving, from the client device, the selection of the first order made based at least on the one or more orders output at the client devices.

In some variations, the patient encounter command and the selection of the first order may include one or more voice inputs, text inputs, and/or haptic inputs received at the client device.

In some variations, the scannable token may be generated to include a uniform resource locator (URL) of a server from which the one or more medical devices retrieves at least the portion of the information associated with the first order.

In some variations, the scannable token may be generated to include at least the portion of the information associated with the first order.

In some variations, the information associated with the first order may be encrypted. The scannable token may be further generated to include a uniform resource locator (URL) of a server from which to retrieve a key for decrypting the information.

In some variations, the patient interaction may include a first interaction with a first medical device followed by a second interaction with a second medical device. An error message may be triggered in response to the scannable token being scanned at the second medical device before the first medical device.

In some variations, the scannable token may be further generated for a second patient encounter to fulfill a second order associated with the first patient or a second patient. The first patient encounter may be prioritized over the second patient encounter based on a scheduled time of each encounter, a first location of the clinician relative to a second location of the first patient and a third location of the second patient, a shift and break scheduling of the clinician, and/or a criticality of each patient.

In some variations, the first patient encounter and the second patient encounter may be conducted by a same clinician or different clinicians.

In some variations, the operations may further include: in response to the scannable token being scanned at the one or more medical devices, sending, to the client device, one or more instructions, reminders, and/or feedback associated with the encounter.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to interconnecting medical devices involved in a clinical workflow, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating a virtual assistant system, in accordance with some example embodiments;

FIG. 2A depicts a schematic diagram illustrating an example of a clinical workflow, in accordance with some example embodiments;

FIG. 2B depicts a schematic diagram illustrating another example of a clinical workflow, in accordance with some example embodiments;

FIG. 2C depicts a schematic diagram illustrating an example of a clinical workflow, in accordance with some example embodiments;

FIG. 3 depicts a sequence diagram illustrating an example of a process for conducting a scannable token-based patient encounter, in accordance with some example embodiments;

FIG. 4A depicts a flowchart illustrating an example of a process for conducting a scannable token-based patient encounter, in accordance with some example embodiments;

FIG. 4B depicts a flowchart illustrating another example of a process for conducting a scannable token-based patient encounter, in accordance with some example embodiments; and

FIG. 5 depicts a block diagram illustrating a computing system, in accordance with some example embodiments.

When practical, similar reference numbers denote similar structures, features, or elements.

### DETAILED DESCRIPTION

In order to administer care to various patients, a clinician may interact with a variety of medical devices including, for example, dispensing cabinets, infusion pumps, wasting stations, and/or the like. Nevertheless, existing medical devices have limited interconnectivity. That is, despite the fact that most clinical workflows require interactions with a sequence of medical devices, critical data for conducting each clinical workflow, such as patient, prescription, or therapy information, is not shared across different medical devices. Consequently, to administer a medication to a patient, a clinician may be required to input patient information when dispensing the medication from a dispensing cabinet before the same patient information is input again to configure an infusion pump to deliver the medication and/or to waste any unused medication at a wasting station. The resulting workflow may lack efficiency, thwart patient engagement, and distract clinicians from actual patient care._{[A1]}

In some example embodiments, a virtual assistant may interconnect different medical devices involved in an encounter with a patient. The virtual assistant leverages the existing functionalities of various medical devices and can therefore be implemented with minimal additions and modifications to the hardware infrastructure of a medical facility. For example, upon receiving information associated with the patient encounter, an assistant server may generate a token for interacting with one or more medical devices involved in the encounter. The token may be scanned at a first medical device involved in the encounter (e.g., a dispensing cabinet) in order to dispense one or more medications for administering to the patient before being scanned at a second medical device involved in the encounter (e.g., an infusion pump) in order to configure the second medical device for delivering the one or more medications to the patient. In some cases, disposing any unused portions of the one or more medications at a third medical device involved in the encounter (e.g., a wasting station) may include scanning the token at the third medical device.

In some example embodiments, the token generated at the assistant server may be displayed at an assistant client (e.g., a mobile device running an assistant application) of a clinician conducting the encounter with the patient. In this case, the token may be scanned at each of the medical devices involved in the encounter. Alternatively or additionally, the token generated at the assistant server may be displayed at the medical devices involved in the encounter, in which case the scanning of the token may be performed by the assistant client of the clinician conducting the encounter with the patient. Scanning the token at a medical device may trigger, at the medical device, one or more actions associated with the encounter. In cases where the token is associated with a limited lifespan, scanning the token at a medical device after the expiration of the token may trigger an error message but not any of the actions associated with the encounter.

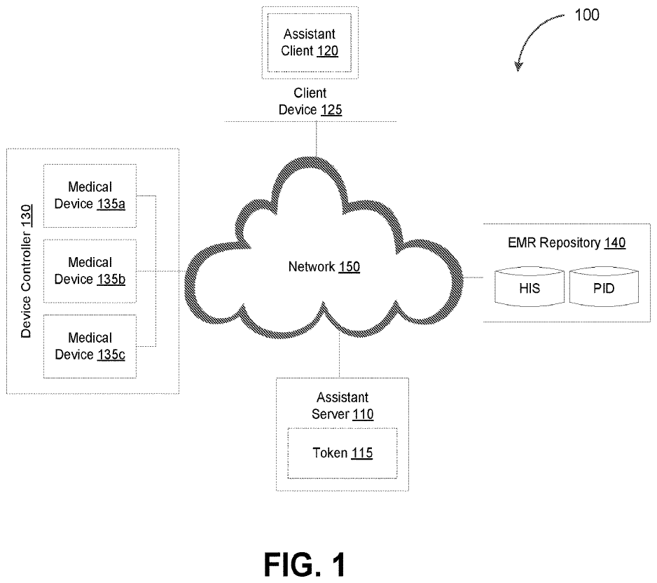

FIG. 1 depicts a system diagram illustrating a virtual assistant system 100, in accordance with some example embodiments. Referring to FIG. 1, the virtual assistant system 100 may include an assistant server 110, an assistant client 120 deployed at a client device 125, a device controller 130 associated with one or more medical devices 135, and an electronic medical record (EMR) repository 140. As FIG. 1 shows, the assistant server 110, the client device 125, the medical device controller 130, and the electronic medical record repository 140 may be communicatively coupled via a network 150.

In the example shown in FIG. 1, the client device 125 may be a specifically configured processor-based device such as, for example, a smartphone, a tablet computer, a wearable apparatus, a desktop computer, a laptop computer, a workstation, and/or the like. The assistant client 120 may be an application, such as a mobile application or a web application, running on the client device 125 to perform one or more of the features described. In some cases, the client device 125 including the assistant client 120 may be a wireless handheld terminal specifically configured for the management of medical care in a clinical environment such as a hospital. The client device 125 may be (or include) a barcode module (BCM) capable of displaying and scanning codes corresponding to, for example, patient identification, item identification, documentation characters and phrases, commands, and instructions. The codes are preferably machine-readable codes, including one and two dimensional optically readable codes such as bar codes, but can include radio frequency identification (RFID) and near field communication (NFC) devices or tags. The codes can be applied to objects, cards, or placards throughout a hospital environment. Alternatively or additionally, as will be described in more detail below, the codes may be a token generated by the assistant server 110 and disseminated for display at the client device 125 and/or the one or more medical devices 135.

The one or more medical devices 135 associated with the device controller 130 may include medical devices configured to perform a variety of clinical tasks such as, for example, dispensing, infusion, wasting, vascular access management, urinary output monitoring, supply logistics, pharmacy compounding, diversion detection, and/or the like. Examples of the electronic medical record repository 140 include a hospital information system (HIS) and a patient information database (PID). Meanwhile, the network 150 may be a wired and/or wireless network including, for example, a public land mobile network (PLMN), a local area network (LAN), a virtual local area network (VLAN), a wide area network (WAN), the Internet, and/or the like.

In some example embodiments, the virtual assistant 120 at the client device 125 may be configured to interconnect_{[A2]} the one or more medical devices 135 involved in an encounter with a patient. Upon receiving information associated with the patient encounter, the assistant server 110 may generate a token 115 for interacting with the one or more medical devices 135 involved in the encounter. For example, scanning the token 115 at each of the one or more medical devices 135 may trigger one or more corresponding actions associated with the encounter. In cases where the token 115 is associated with a limited lifespan, scanning the token 115 at the one or more medical devices 135 after the expiration of the token 115 may trigger an error message but not any of the actions associated with the encounter. In such cases, the clinician may still rely on standard workflows to interact with the device, but, as discussed, this may take more time or actions than when the token 115 is valid.

In some example embodiments, the token 115 may be associated with a limited lifespan corresponding to an estimated or expected quantity of time required to complete the encounter with the patient. Accordingly, in some cases, the lifespan of the token 115 may be determined based on one or more factors such as the location of the clinician relative to the location of the patient associated with each encounter, the nature of the tasks associated with each encounter, and the like. The token 115 may therefore be assigned a longer lifespan if the token 115 is associated with more complex tasks or a patient at a more distant location.

In some example embodiments, the status of the token 115 may transition based on whether the token 115 is scanned before its expiration. For example, the token 115 may expire without ever having been scanned. In some cases, the token 115 may expire once the assistant server 110 determines that the corresponding encounter is complete even though the token 115 was not scanned at any of the medical devices 135 involved in the encounter. Determining the encounter is complete may be based on information received from the client device, electronic medical records system, patient data management system, or other device accessible by the assistant server 110. Alternatively, in other cases, the token 115 may be scanned at least once but may expire before the encounter is complete. In some cases, the assistant server 110 may determine to extend the lifespan of the token 115 in response to the token 115 being scanned a threshold quantity of times and/or at a threshold quantity of medical devices, for example, prior to the expiration of the token 115_{[A3][A4]}. For example, the assistant server 110 may dynamically determine, based at least on Equation (1) below, a quantity of time extension applied to the lifespan of the token 115. In accordance with Equation (1), the quantity of time extension applied to the lifespan of the token 115 may correspond to the proximity between a current medical device (e.g., the first medical device 135a) and a next medical device (e.g., the second medical device 135b) in the encounter. Moreover, in some instances, the frequency with which the tokens generated for a particular clinician expire before being scanned or expire before completion of the corresponding encounter may serve as data points for assessing the likelihood of the clinician engaging in hazardous behavior such as diversion.

$$TimeExtension = dist ( current to next ) \times avg\_pace + buffer \qquad ( 1 )$$

wherein dist(current to next) denotes the distance between the current medical device and the next medical device in an encounter, avg_pace denotes the average travel speed (e.g., actual speed of the clinician associated with the token 115, speed derived from historical activities (e.g., scans), and/or the like), and buffer denotes a system, user, or dynamically generated time buffer to account for random or variability in the clinical environment.

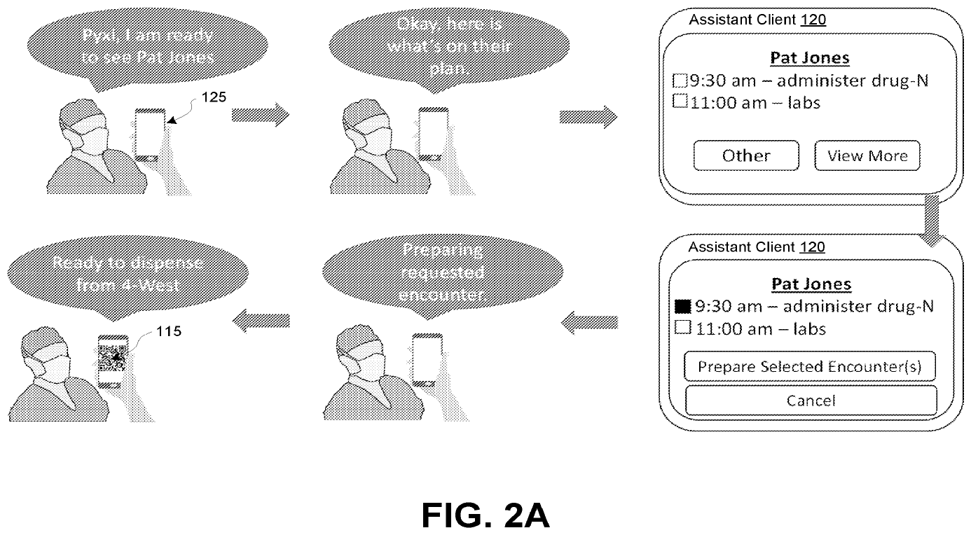

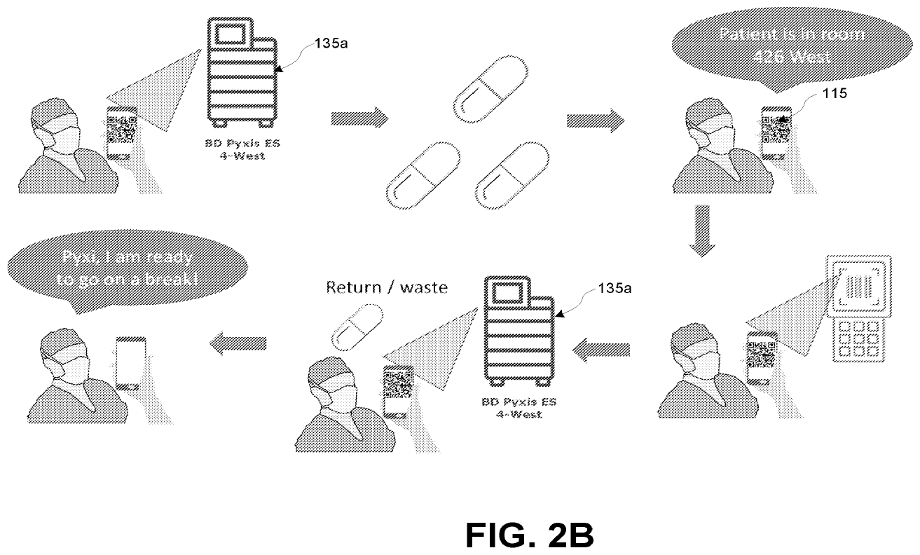

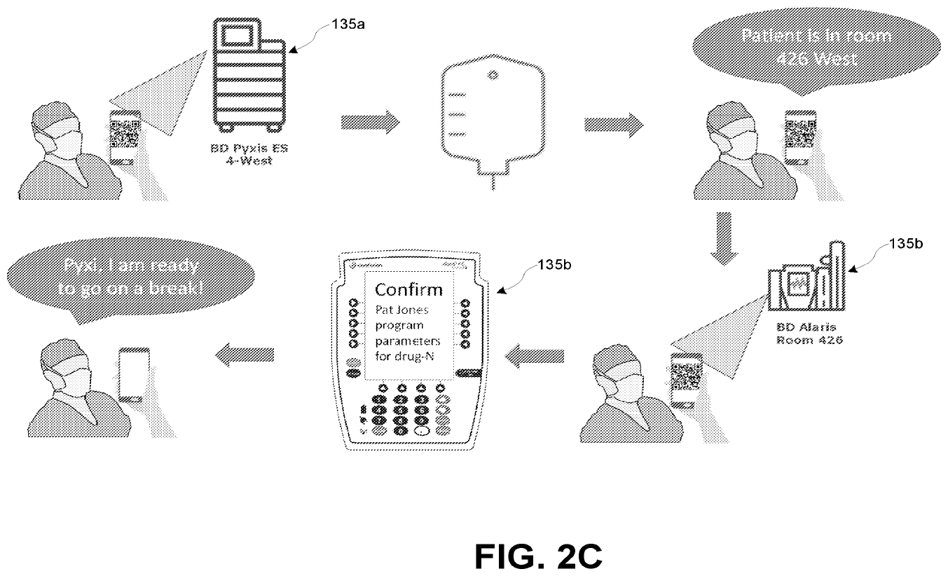

To further illustrate, FIG. 2A depict one example of a clinical workflow 200 conducted via the assistant client 120 at the client device 125. In some example embodiments, a clinician may interact with the assistant client 120 at the client device 125 via, for example, voice inputs, text inputs, haptic inputs, and/or the like. To process voice inputs, the assistant server 110 and/or the assistant client 120 may apply one or more natural language processing (NLP) techniques. Referring to FIG. 2A, the clinical workflow 200 may include the assistant client 120 responding to a first user input from the clinician specifying a patient (e.g., Pat Jones) by at least displaying, at the client device 125, one or more encounters associated with the patient (e.g., administer drug N at 9:30 AM, labs at 11:00 AM, and/or the like). In some example embodiments, the assistant client 120 may provide a recommended sequence for conducting the one or more encounters, for example, by displaying the one or more encounters in an order corresponding to the priority of each encounter. The respective priority of each encounter may in turn may be determined based on a variety of factors including, for example, the scheduled time of each encounter, the location of the clinician relative to the location of the patient associated with each encounter, the shift and break scheduling of the clinician, the criticality of each patient, and/or the like.

As shown in FIG. 2A, the assistant client 120 may receive a second user input selecting one of the encounters associated with the patient (e.g., administer drug N at 9:30 AM). In response to the second user input selecting one of the encounters associated with the patient, the assistant client 120 may prepare the selected encounter which may include interacting with the assistant server 110 to generate the token 115 associated with the encounter. As shown in FIG. 2A, the token 115, which the assistant client 120 may receive from the assistant server 110, may be displayed at the client device 125 such that the token 115 may be used to conduct the encounter. In some cases, selecting a particular encounter may include entering the client device 125 in a remote queue for accessing the one or more medical devices 135 associated with the encounter. For example, in some example embodiments, the client device 125 including the assistant client 120 may automatically connect to the first medical device 135a to enter an order for the medication (e.g., drug N). This connection may be established irrespective of where the medication is actually stored, and without the need to queue up with other clinicians at a single terminal. In some cases, the pairing between the client device 125 and the first medical device 135a may prevent, or lock out, other clinicians from pairing to or accessing the first medical device 135 for the duration of the pairing while in other cases, the first medical device 135a may support multiple pairings but restrict access to medication to a single device (e.g., the currently paired client device 125) based on a software queue.

FIG. 2B depicts another example of a clinical workflow in which the token 115 is used to conduct an encounter with a patient. For example, in some cases, the token 115, which the assistant client 120 may receive from the assistant server 110, may be displayed at the client device 125 such that the token 115 may be presented at the first medical device 135a involved in the encounter with the patient. In the example shown in FIG. 2B, the first medical device 135a is a dispensing cabinet from which the medication (e.g., drug N) for administering to the patient (e.g., Pat Jones) during the encounter may be dispensed by scanning the token 115 displayed at the client device 125. However, it should be appreciated that in some cases, the medication may be retrieved from the first medical device 135a as a part of the discharge procedure for the patient. In some cases, the token 115 may be scanned as a part of authenticating the clinician associated with the client device 125. For instance, the token 115 may encode an authentication token associated with the clinician and/or serve as an additional authentication factor for verifying the identity of the clinician. In such instances, the authentication process may be adjusted based on whether a valid token 115 is presented at the first medical device 135a.

Furthermore, in some example embodiments, scanning the token 115 at the first medical device 135a may trigger one or more actions associated with the dispensing of the medications for the patient including, for example, the unlocking of the receptacles (e.g., drawers, pockets, and/or the like) holding the medication, the generation of one or more electronic records documenting the transaction, and/or the like. The one or more records for the transaction may include one or more transaction values corresponding to, for example, timestamps, patient identifiers, device identifiers, clinician identifiers, medication identifiers, prescription order identifiers, inventory information, patient status, shift identifiers, location tracking identifiers, infusion information, compounding information, administration information, working off clock indicators, electronic health record (EHR) identifiers, and/or the like.

Referring again to FIG. 2B, upon retrieving the medication (e.g., drug N) from the first medical device 135a, the assistant client 120 may provide, through the client device 125, one or more instructions, reminders, and/or feedback associated with the encounter. In the example shown in FIG. 2B, the assistant client 120 may display, at the client device 125, one or more locations at which the patient (e.g., Pat Jones) requiring the medication retrieved from the first medical device 135a may be located. The assistant client 120 may also display, at the client device 125, procedural instructions associated with the first medical device 135a or the medication being administered to the patient. In some cases, the assistant client 120 may display, at the client device 125, encouragements and patient specific instructions such as the patient's birthday, previous complications, allergies, and/or the like. It should be appreciated that the assistant client 120 may use a variety of different modalities, including text messages, voice prompts, haptic feedback, push notifications, and/or the like, in order to provide the one or more instructions, reminders, and/or feedback. Moreover, in some cases, the assistant client 120 may provide the one or more instructions, reminders, and/or feedback associated with the encounter proactively, for example, through push notifications, text messages, and/or the like, without any triggers from the clinician.

In some example embodiments, once the medication retrieved from the first medical device 135a is administered to the patient, the assistant client 120 may provide a variety of mechanisms to confirm and document the administration of the medication. For example, the assistant client 120 at the client device 125 may provide one or more user interface elements configured to receive one or more user inputs confirming the administration of the medication. Accordingly, in some cases, the administration of the medication may be confirmed based on user inputs the assistant client 120 receives through the client device 125 (e.g., one or more corresponding user interface elements displayed at the client device 125). Alternatively or additionally, the administration of the one or more medication may be confirmed by scanning, at an electronic medical record (EMR) terminal, the token 115 displayed by the assistant client 120 at the client device 125.

Referring again to FIG. 2B_{[A5][A6]}, after administering the medication and, in some instances, confirming the administration of the medication through the assistant client 120, the encounter with the patient may continue with the return of any unused portions of the medication. In the example shown in FIG. 2B, unused portions of the medication retrieved from the first medical device 135a may be returned to the first medical device 135a (or a different medical device) for wasting and/or disposal. Moreover, as shown in FIG. 2B, the return of the unused medication may be performed by scanning, at the first medical device 135a, the token 115 in order to trigger one or more actions associated with the return, wasting, and/or disposal of the unused medications. Examples of such actions may include unlocking a receptacle (e.g., a drawer, a pocket, and/or the like) designated for receiving the unused medication, the generation of one or more electronic records documenting the transaction, and/or the like. The one or more records for the transaction may include one or more transaction values corresponding to, for example, timestamps, patient identifiers, device identifiers, clinician identifiers, medication identifiers, prescription order identifiers, inventory information, patient status, shift identifiers, location tracking identifiers, infusion information, compounding information, administration information, working off clock indicators, electronic health record (EHR) identifiers, and/or the like. Once the unused medication is returned to the first medical device 135a, the encounter with the patient may terminate. The trigger may be based on comparison of the token 115 or data encoded thereby with the one or more records. For example, the token 115 may include an identifier for the medication to be returned. Without a token, the first medical device 135a may require a user to log into the device and authenticate themselves. From there, the first medical device 135a may need to be configured to perform a wasting workflow which may also include additional steps to identify the drug, quantity to waste, etc. However, using a token, upon presentation to the first medical device 135a, the device 135a may skip one or more of the workflow steps based on the information associated with the token 115. In addition to saving time, the token 115 can also reduce error in configuring the first medical device 135a (e.g., avoiding manual data entry error, proper workflow selection, etc.).

FIG. 2C_{[A7][A8]} depicts another example of a clinical workflow conducted via the assistant client 120 at the client device 125. In this example, instead of the medication being administered orally to the patient, the encounter with the patient (e.g., Pat Jones) may include the intravenous administration of the medication (e.g., drug N). Accordingly, upon scanning the token 115 to retrieve the medication from the first medical device 135a (e.g., a dispensing cabinet, a supply room, and/or the like), the token 115 may subsequently be scanned at a second medical device 135b, which in this example is an infusion pump configured to deliver the medication retrieved from the first medical device 135a. As shown in FIG. 2C, the second medical device 135b may serve as the barcode module (BCM) such that the token 115 is displayed at the client device 125 and scanned by the second medical device 135b. Alternatively, in some example embodiments, it may also be possible for the client device 125 to serve as the barcode module (BCM) meaning that the token 115 is displayed at the second medical device 135b and scanned by the client device 125 instead. In either case, the scanning of the token 115 may trigger, at the second medical device 135b, one or more actions associated with the delivery of the medication retrieved from the first medical device 135a. For instance, FIG. 2C shows that the scanning of the token 115 may configure the second medical device 135b with infusion parameters for delivering the medication (e.g., drug N) to the patient (e.g., Pat Jones). The patient encounter may terminate once the medication retrieved from the first medical device 135a is delivered to the patient using the second medical device 135b. Alternatively, although not shown in FIG. 2C, the patient encounter may terminate after unused portions of the medication are returned to the first medical device 135a (or a different medical device), for example, by scanning the token 115 at the first medical device 135a.

Although FIGS. 2A-C show examples of clinical workflows in which the token 115 is associated with a single patient encounter (e.g., a single order for a single patient), it should be appreciated that in some cases, the token 115 may be generated for multiple encounters associated with the same patient or different patients and conducted by the same clinicians or different clinicians. For example, the token 115 may be associated with encounters with multiple patients conducted by the same clinician. The token 115 can also be associated with multiple encounters associated with the same patient. Alternatively or additionally, the token 115 may be associated with multiple patient encounters that occur at a certain time (e.g., 9 AM med pass). When the token 115 is associated with multiple patient encounters, the encounters may be prioritized, as noted earlier, based on factors such as the scheduled time of each encounter, the location of the clinician relative to the location of the patient associated with each encounter, the shift and break scheduling of the clinician, the criticality of each patient, and/or the like.

As noted, the assistant client 120 at the client device 125 may interact with the assistant server 110 in order to generate the token 115 for interacting with the one or more medical devices 135. In some example embodiments, the assistant server 110 may further interact with the device controller 130 and the electronic medical record repository 140 in order to consolidate critical data that is typically isolated at the device controller 130 and the electronic medical record repository 140. In doing so, the assistant server 110 may generate the token 115 to incorporate patient data from the electronic medical record repository 140 and device information from the device controller 130 when generating the token 115. For example, the resulting token 115 may include, for each patient encounter, information for the corresponding order, patient, clinician, and/or the like.

In some cases, the token 115 may be generated to encode a unique identifier including information for locating the assistant server 110 (e.g., a uniform resource locator (URL) of the assistant server 110) such that the corresponding information may be retrieved, based on the unique identifier, from the assistant server 110 may the assistant client 120 at the client device 125 and/or the medical devices 135. For example, a first portion of the identifier may include a network address of the assistant server 110 and a second portion may include a character string associated with the encounter. The character string, when presented to the assistant server 110, may cause the assistant server 110 to transmit a response message including information for the encounter. Alternatively, the token 115 may be generated to include, for each patient encounter, the information required by each of the medical devices 135 involved in the encounter (e.g., order, patient, and clinician information). In such instances, the token 115 may include delimited data elements parsable by each device. When presented to a specific device, the device may extract data from the fields it needs for the interaction. In some cases, the information included in the token 115 may include a specific sequence of tasks and the corresponding medical devices. For example, the token 115 may encode information specifying that the encounter includes a first interaction with the first medical device 135a followed by a second interaction with the second medical device 135b, in which case scanning the token 115 at a third medical device 135c after the token 115 is scanned at the first medical device 135a may trigger a corresponding error message. It should be appreciated that the information included in the token 115 may be encrypted. Moreover, in some cases, the token 115 may still include the unique identifier of the assistant server 110 (e.g., a uniform resource locator (URL) of the assistant server 110) but instead of retrieving information from the assistant server 110, the assistant client 120 may retrieve a key for decrypting the encrypted information included in the token 115.

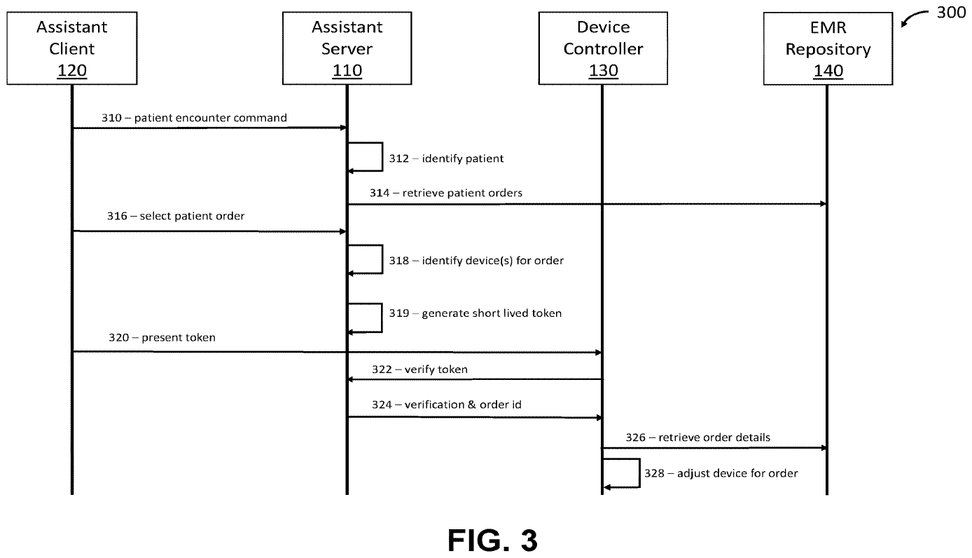

To further illustrate, FIG. 3 depicts a sequence diagram illustrating an example of a process 300 for conducting a scannable token-based patient encounter, in accordance with some example embodiments. Referring to FIG. 3, at 310, the assistant client 120 at the client device 125 may send, to the assistant server 110, a patient encounter command. In some cases, the patient encounter command may specify a particular patient (e.g., Pat Jones) by specifying one or more patient identifiers (e.g., patient name, patient identification number, and/or the like). In response to the patient encounter command, the assistant server 110 may identify the patient at 312. Identifying the patient may include natural language processing an audio of an utterance associated with the encounter command. Identifying the patient may include decoding or looking up a patient identifier based on the encounter command. At 314, the assistant server 110 may retrieve, based at least on the information identifying the patient, one or more patient orders from the electronic medical record (EMR) repository 140. Referring again to FIG. 2A, examples of patient orders may include the administration of a medication (e.g., drug N), lab tests, physical therapy, or the like. Moreover, each patient order (e.g., administer multiple drug orders, collect specimen for lab test, and physical therapy motion exercise) may be associated with an encounter with the patient.

One or more of the patient orders retrieved from the electronic medical record repository 140 may be displayed, for example, by the assistant client 120 at the client device 125. The assistant client 120 may receive a user input selecting one of the orders displayed at the client device 125. The selected order may be sent to the assistant server 110 at 316, thus initiating a clinical workflow for conducting the patient encounter corresponding to the selected order. For example, at 318, the assistant server 110 may identified the one or more medical devices 135 required to fulfill the order. Identification may be based on the order type (e.g., medication administration). The order type may be associated with a dispensing device, an administration device, and a wasting or return device. The association may be stored in a data storage accessible by the assistant server 110 and indexed using, for example, order type. Moreover, at 319, the assistant server 110 may generate a token (e.g., a token with a limited lifespan) for interacting with the one or more medical devices 135 required to fulfill the order. The token may be generated to encode information and/or enable the retrieval of information required for conducting the encounter at each of the one or more medical devices for the encounter (e.g., those identified at 318). It should be appreciated that operation 318 may be optional in cases where the one or more medical devices 135 are configured to contact the assistant server 110 in response to the scanning of the token 115.

As noted, the assistant client 120 may display the token 115 at the client device 125 such that the token 115 may be scanned at the one or more medical devices 135. In the example shown in FIG. 3, the token may be presented to the device controller 130 at 320 before the device controller 130 verifies the token 115 at 322. Verification of the token 115 may include determining whether the token 115 is not expired, is associated with an authorized user of the medical device, or the like. At 324, the device controller 130 may verify the order identifier associated with the token 115 before retrieving the corresponding order details from the electronic medical record (EMR) repository 140 at 326. For example, an order may change between when the token 115 was generated and when presented to the medical device. Similarly, the verification at 324 may be used as a security check to authorize activity via the device controller 130. Once confirmed, the device controller 130 may use at least a portion of the order information to retrieve details needed to administer the order. At 328, the device controller 130 may configure the one or more medical devices 135 in accordance with the details of the order retrieved from the electronic medical record repository 140. For instance, as shown in FIG. 2C, the device controller 130 may configure the second medical device 135b in accordance with the infusion parameters associated with the patient (e.g., Pat Jones) and the medication being delivered (e.g., drug N).

As used herein, the terms “adjust” or “adjusting” or “control” or “controlling” may encompass a wide variety of actions. For example, “adjusting” a medical device may include transmitting one or more messages to change an operational state or functional element of the device. The message may include specific instructions to be executed by a processor of the device to manifest the change. The “adjusting” may include storing a value in a location of a storage device for subsequent retrieval by the device to be controlled, transmitting a value directly to the device to be controlled via at least one wired or wireless communication medium, transmitting or storing a reference to a value, and the like. For example, a control message may include a value to adjust a level of power from a power source of the controlled device. As another example, a control message may activate or deactivate a structural element of the controlled device such as a light, audio playback, a motor, a lock, a pump, a dispensing cabinet, a dispensing drawer, a dispensing bin, a display, or other component of a device described herein. “Controlling” or “adjusting” may include selecting one of a set of workflows at a device. For example, a medication return device may be configurable to allow returns in one manner for fluid drugs and in a second manner for solid drugs. The adjustment in such a case may activate different workflow and hardware to facilitate safe and verifiable collection of the material type. “Controlling” or “adjusting” may include indirect control of the device by adjusting a configuration value used by the controlled device. For example, the control message may include a threshold value for a device characteristic (e.g., temperature, rate, frequency, etc.). The threshold value may be stored in a memory location and referred to by the controlled device during operation.

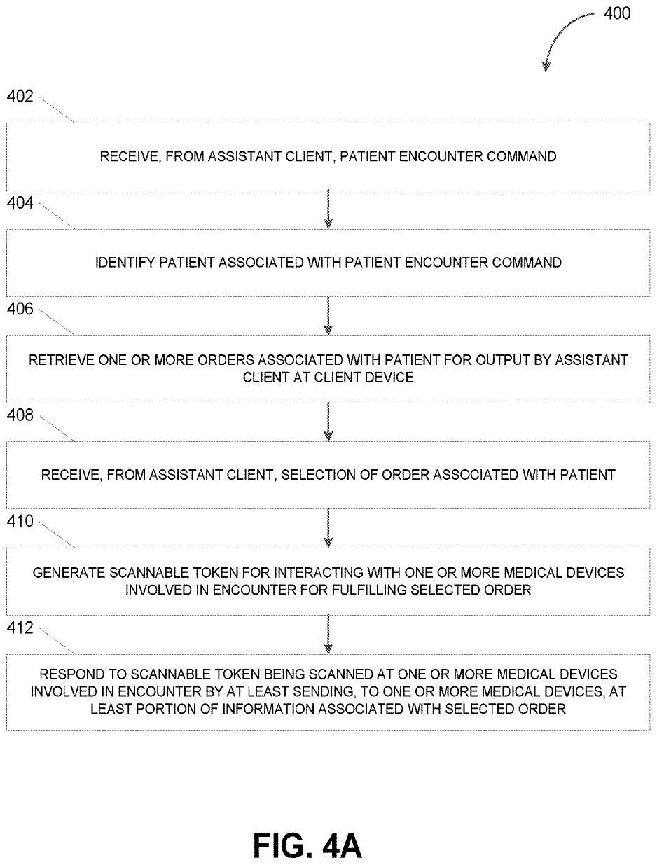

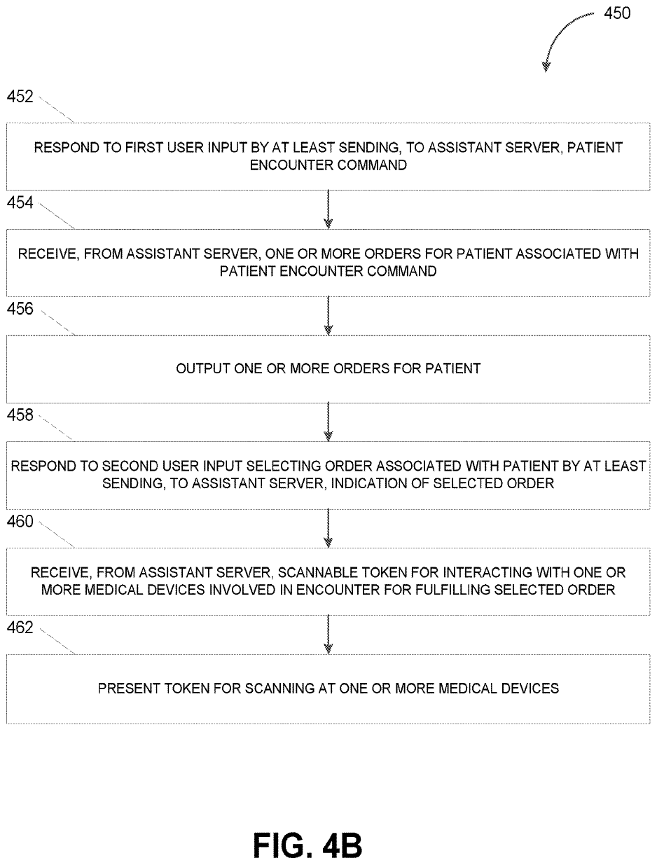

FIG. 4A depicts a flowchart illustrating an example of a process 400 for conducting a scannable token-based patient encounter, in accordance with some example embodiments. Referring to FIGS. 1, 3, and 4A, the process 400 may be performed by the assistant server 110 in order to conduct a patient encounter involving one or more of the medical devices 135.

At 402, the assistant server 110 may, from the assistant client 120, receive a patient encounter command. For example, the assistant server 110 may receive, from the assistant client 120 at the client device, a patient encounter command to initiate an encounter with a specific patient (e.g., Pat Jones). The patient encounter command may specify the patient by specifying one or more patient identifiers such as patient name, patient identification number, and/or the like.

At 404, the assistant server 110 may identify a patient associated with the patient encounter command. For example, the assistant server 110 may identify, based at least on the one or more patient identifiers included in the patient encounter command, a corresponding patient.

At 406, the assistant server 110 may retrieve one or more orders associated with the patient for output by the assistant client 120 at the client device 125. In some example embodiments, such as shown in FIG. 2A, the assistant server 110 may retrieve, from the electronic medical record (ERM) repository 140, one or more outstanding orders associated with the patient (e.g., administer drug N at 9:30 AM, labs at 11:00 AM, and/or the like). In the example shown in FIG. 2A, these orders may be displayed by the assistant client 120 at the client device 125.

At 408, the assistant server 110 may receive, from the assistant client 120, a selection of an order associated with the patient. For instance, in the example shown in FIG. 2A, the assistant server 110 may receive, from the assistant client 120 at the client device 125, a selection of the order to administer drug N to the patient. The selection may include multiple orders for the same or different patients.

At 410, the assistant server 110 may generate a scannable token for interacting with one or more medical devices involved in an encounter for fulfilling the selected order. In some example embodiments, the assistant server 110 may generate the token 115 for interacting with the one or more medical devices 135 involved in an encounter to fulfill the order to administer drug N to the patient. For example, FIG. 2B shows one example workflow where the encounter to fulfill the order to administer drug N to the patient include interacting with the first medical device 135a (e.g., a dispensing cabinet) to retrieve drug N and to return unused portions of drug N. Alternatively, FIG. 2C shows another example workflow where the encounter to administer drug N further includes interacting with the second medical device 135b (e.g., an infusion pump) to deliver drug N to the patient. In either instance, the token 115 may be generated to encode information and/or enable the retrieval of information required for conducting the encounter at each of the one or more medical devices 135. For example, the token 115 may be generated to encode a unique identifier for locating the assistant server 110 (e.g., a uniform resource locator (URL) of the assistant server 110) such that the corresponding information may be retrieved, based on the unique identifier, from the assistant server 110 may the assistant client 120 at the client device 125 and/or the medical devices 135. Alternatively, the token 115 may be generated to include, for each patient encounter and in encrypted form, the information required by each of the medical devices 135 involved in the encounter (e.g., order, patient, and clinician information). In some cases, the assistant server 110 may identify the one or more medical devices 135 involved in the encounter before generating the token 115. However, this operation may be omitted in instances where the one or more medical devices 135 are configured to contact the assistant server 110 in response to the scanning of the token 115.

At 412, the assistant server 110 may respond to the scannable token being scanned at the one or more medical devices involved in the encounter by at least sending, to the one or more medical devices, at least a portion of information associated with the selected order. For example, when the token 115 is scanned at the one or more medical devices 135, the assistant server 110 may verify the token 115 before sending, to the device controller 130, the corresponding order identifier such that the device controller 130 may retrieve the corresponding order details from the electronic medical record (EMR) repository 140 and send, to the one or more medical devices 135, the order details for conducting the encounter at each of the one or more medical devices 135. In some cases, the order details sent to each of the one or more medical devices 135 may trigger actions such as configuring the one or more medical devices 135, generating one or more records documenting the corresponding transactions, and/or the like.

FIG. 4B depicts a flowchart illustrating another example of a process 450 for conducting a scannable token-based patient encounter, in accordance with some example embodiments. Referring to FIGS. 1 and 4B, the process 450 may be performed by the assistant client 120 at the client device 125 in order to conduct a patient encounter involving one or more of the medical devices 135.

At 452, the assistant client 120 may respond to a first user input by at least sending, to the assistant server 110, a patient encounter command. For example, as shown in FIG. 2A, the assistant client 120 at the client device 125 may receive a first user input initiating an encounter with a particular patient (e.g., Pat Jones). The assistant client 120 may respond to the first user input by at least sending, to the assistant server 110, a corresponding patient encounter command, which may specify the patient by specifying one or more patient identifiers such as patient name, patient identification number, and/or the like.

At 454, the assistant client 120 may receive, from the assistant server 110, one or more orders for a patient associated with the patient encounter command. For example, the assistant client 120 may receive, from the assistant server 110, the assistant server 110 may retrieve, from the electronic medical record (ERM) repository 140, one or more outstanding orders associated with the patient (e.g., administer drug N at 9:30 AM, labs at 11:00 AM, and/or the like). As noted, the assistant server 110 may retrieve, based on the one or more patient identifiers (e.g., patient name, patient identification number, and/or the like), the one or more outstanding orders from the electronic medical record (EMR) repository 140.

At 456, the assistant client 120 may output the one or more orders for the patient. In some example embodiments, the assistant client 120 may output the one or more orders by at least displaying, in a graphic user interface (GUI) at the client device 125, the one or more orders. Alternatively or additionally, the one or more orders may be provided as a voice output at the client device 125.

At 458, the assistant client 120 may respond to a second user input selecting an order associated with the patient by at least sending, to the assistant server 110, an indication of the selected order. For example, upon receiving a second user input selecting the order to administer drug N to the patient, the assistant client 120 may send, to the assistant server 110, a corresponding indication of the selected order. As noted, the assistant server 110 may respond to receiving the indication of the selected order by at least identifying the one or more medical devices 135 involved in an encounter to fulfill the selected order. Moreover, the assistant server 110 may respond to receiving the indication of the selected order by generating the token 115, which may be used for interacting with the one or more medical devices 135 involved in the encounter.

At 460, the assistant client 120 may receive, from the assistant server 110, a scannable token for interacting with one or more medical devices involved in an encounter for fulfilling the selected order. In some example embodiments, the assistant client 120 may receive, from the assistant server 110, the token 115 for interacting with the one or more medical devices 135 involved in the encounter for delivering drug N to the patient.

At 462, the assistant client 120 may present the token for scanning at the one or more medical devices. In some example embodiments, the token 115 may be generated to encode information and/or enable the retrieval of information required for conducting the encounter at each of the one or more medical devices 135. For example, the token 115 may be generated to encode a unique identifier for locating the assistant server 110 (e.g., a uniform resource locator (URL) of the assistant server 110) such that the corresponding information may be retrieved, based on the unique identifier, from the assistant server 110 may the assistant client 120 at the client device 125 and/or the medical devices 135. Alternatively, the token 115 may be generated to include, for each patient encounter and in encrypted form, the information required by each of the medical devices 135 involved in the encounter (e.g., order, patient, and clinician information). Accordingly, when the token 115 is scanned at the first medical device 135a, for example, the first medical device 135a may determine, based at least on the token 115, information for conducting a corresponding portion of the encounter at the first client device 135a. If the medical device is not configured to read the token 115 or the token 115 is expired or otherwise invalid, the medical device may still be operable to perform the patient encounter. However, at least one step in using the medical device will need to be performed manually that would otherwise be bypassed if the token 115 were valid.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Item 1: A method, comprising: receiving a selection of a first order associated with a first patient; generating a scannable token for interacting with one or more medical devices involved in a first patient encounter to fulfill the first order; sending, to a client device, the scannable token; and in response to the scannable token being scanned at the one or more medical devices, sending, to the one or more medical devices, at least a portion of information associated with the first order.

Item 2: The method of Item 1, wherein the scannable token is associated with a limited lifespan, and wherein the scannable token expires upon the scannable token reaching an end of the limited lifespan without being scanned at the one or more medical devices.

Item 3: The method of Item 2, wherein the limited lifespan of the scannable token is extended in response to the scannable token being scanned a threshold quantity of times and/or at a threshold quantity of the one or more medical devices prior to the expiration of the scannable token.

Item 4: The method of any of Items 1 to 3, wherein the scannable token expires prior to an end of the limited lifespan of the scannable token in response to determining that the first patient encounter is complete without the scannable token being scanned at any of the one or more medical devices.

Item 5: The method of any of Items 1 to 4, further comprising: verifying the scannable token scanned at the one or more medical devices prior to sending, to the one or more medical devices, at least the portion of the information associated with the first order.

Item 6: The method of any of Items 1 to 5, further comprising: authenticating, based at least on the scannable token scanned at the one or more medical devices, an identity of a clinician interacting with the one or more medical devices.

Item 7: The method of Item 6, wherein the scannable token comprises an additional authentication factor for authenticating the identity of the clinician interacting with the one or more medical devices.

Item 8: The method of any of Items 1 to 7, wherein at least the portion of the information sent to the one or more medical devices include one or more parameters to configure the one or more medical devices for the first order.

Item 9: The method of any of Items 1 to 8, wherein the one or more medical devices include an infusion pump, and wherein at least the portion of the information sent to the one or more medical devices include one or more infusion parameters associated the first patient and/or a medication being delivered to the first patient.

Item 10: The method of any of Items 1 to 9, wherein the one or more data medical devices include a dispensing cabinet, and wherein at least the portion of the information sent to the one or more medical devices unlocks a first receptacle from which to retrieve a medication included in the first order and/or a second receptacle for returning an unused portion of the medication after the medication is administered to the first patient.

Item 11: The method of any of Items 1 to 10, wherein at least the portion of the information sent to the one or more medical devices triggers a generation of one or more electronic records documenting an interaction with the one or more medical devices to fulfill the first order.

Item 12: The method of any of Items 1 to 11, further comprising: receiving a patient encounter command specifying the first patient; in response to the patient encounter command, retrieving, from an electronic medical record (EMR) repository, one or more orders associated with the first patient; sending, to the client device, the one or more orders for output at the client device; and receiving, from the client device, the selection of the first order made based at least on the one or more orders output at the client devices.

Item 13: The method of any of Items 1 to 12, wherein the patient encounter command and the selection of the first order comprise one or more voice inputs, text inputs, and/or haptic inputs received at the client device.

Item 14: The method of any of Items 1 to 13, wherein the scannable token is generated to include a uniform resource locator (URL) of a server from which the one or more medical devices retrieves at least the portion of the information associated with the first order.

Item 15: The method of any of Items 1 to 14, wherein the scannable token is generated to include at least the portion of the information associated with the first order.

Item 16: The method of Item 15, wherein the information associated with the first order is encrypted, and wherein the scannable token is further generated to include a uniform resource locator (URL) of a server from which to retrieve a key for decrypting the information.

Item 17: The method of any of Items 1 to 16, wherein the patient interaction includes a first interaction with a first medical device followed by a second interaction with a second medical device, and wherein an error message is triggered in response to the scannable token being scanned at the second medical device before the first medical device.

Item 18: The method of any of Items 1 to 17, wherein the scannable token is further generated for a second patient encounter to fulfill a second order associated with the first patient or a second patient, and wherein the first patient encounter is prioritized over the second patient encounter based on a scheduled time of each encounter, a first location of the clinician relative to a second location of the first patient and a third location of the second patient, a shift and break scheduling of the clinician, and/or a criticality of each patient.

Item 19: The method of Item 18, wherein the first patient encounter and the second patient encounter are conducted by a same clinician or different clinicians.

Item 20: The method of any of Items 1 to 19, further comprising: in response to the scannable token being scanned at the one or more medical devices, sending, to the client device, one or more instructions, reminders, and/or feedback associated with the encounter.

Item 21: A system, comprising: at least one data processor; and at least one memory storing instructions which, when executed by the at least one data processor, result in operations comprising the method of any of Items 1 to 20.

Item 22: A non-transitory computer readable medium storing instructions, which when executed by at least one data processor, result in operations comprising the method of any one of items 1 to 20.

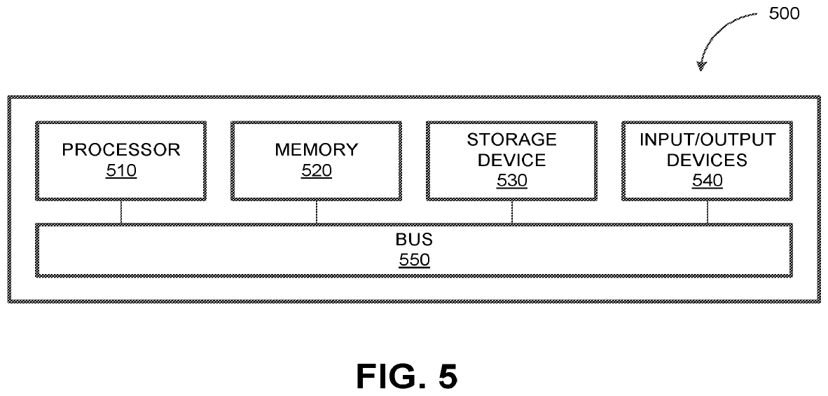

FIG. 5 depicts a block diagram illustrating a computing system 500 consistent with implementations of the current subject matter. Referring to FIGS. 1 and 5, the computing system 500 can be specifically configured to implement the assistant server 110, the client device 120, the device controller 130, and/or components therein.

As shown in FIG. 5, the computing system 500 can include a processor 510, a memory 520, a storage device 530, and input/output device 540540. The processor 510, the memory 520, the storage device 530, and the input/output device 540 can be interconnected via a system bus 550. The processor 510 is capable of processing instructions for execution within the computing system 500. Such executed instructions can implement one or more components of, for example, the assistant server 110, the client device 120, the device controller 130, and/or the like. In some example embodiments, the processor 510 can be a single-threaded processor. Alternatively, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, a tape device, a solid-state device, and/or other suitable persistent storage means. The input/output device 540 provides input/output operations for the computing system 500. In some example embodiments, the input/output device 540 includes a keyboard and/or pointing device. In various implementations, the input/output device 540 includes a display unit for displaying graphical user interfaces.

According to some example embodiments, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example embodiments, the computing system 500 can be specifically configured to execute various interactive computer software applications that can be used for organization, analysis and/or storage of data in various formats. These applications can be used to perform various functionalities, e.g., planning functionalities (e.g., generating, managing, editing of spreadsheet documents, word processing documents, and/or other objects, etc.), computing functionalities, communications functionalities, etc. in support of the encounter and medical device configuration features described. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein can be realized in specifically configured digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to a computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to a signal used to provide machine instructions and/or data to a programmable processor. The machine-readable medium can store such machine instructions non-transitorily, such as for example as would a non-transient solid-state memory or a magnetic hard drive or equivalent storage medium. The machine-readable medium can alternatively or additionally store such machine instructions in a transient manner, such as for example, as would a processor cache or other random-access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be sensory feedback, such as for example visual feedback, auditory feedback, or tactile feedback; and input from the user may be received, such inputs including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions which, when executed by the at least one data processor, result in operations comprising:
receiving a selection of a first order associated with a first patient;
generating a scannable token for interacting with one or more medical devices involved in a first patient encounter to fulfill the first order;
sending, to a client device, the scannable token; and
in response to the scannable token being scanned at the one or more medical devices, sending, to the one or more medical devices, at least a portion of information associated with the first order.

2. The system of claim 1, wherein the scannable token is associated with a limited lifespan, and wherein the scannable token expires upon the scannable token reaching an end of the limited lifespan without being scanned at the one or more medical devices.

3. The system of claim 2, wherein the limited lifespan of the scannable token is extended in response to the scannable token being scanned a threshold quantity of times and/or at a threshold quantity of the one or more medical devices prior to the expiration of the scannable token.

4. The system of claim 3, wherein the scannable token expires prior to an end of the limited lifespan of the scannable token in response to determining that the first patient encounter is complete without the scannable token being scanned at any of the one or more medical devices.

5. The system of claim 4, wherein the operations further comprise:
verifying the scannable token scanned at the one or more medical devices prior to sending, to the one or more medical devices, at least the portion of the information associated with the first order.

6. The system of claim 1, wherein the operations further comprise:
authenticating, based at least on the scannable token scanned at the one or more medical devices, an identity of a clinician interacting with the one or more medical devices.

7. The system of claim 6, wherein the scannable token comprises an additional authentication factor for authenticating the identity of the clinician interacting with the one or more medical devices.

8. The system claim 7, wherein at least the portion of the information sent to the one or more medical devices include one or more parameters to configure the one or more medical devices for the first order.

9. The system of claim 8, wherein the one or more medical devices include an infusion pump, and wherein at least the portion of the information sent to the one or more medical devices include one or more infusion parameters associated the first patient and/or a medication being delivered to the first patient.

10. The system of claim 9, wherein the one or more data medical devices include a dispensing cabinet, and wherein at least the portion of the information sent to the one or more medical devices unlocks a first receptacle from which to retrieve a medication included in the first order and/or a second receptacle for returning an unused portion of the medication after the medication is administered to the first patient.

11. The system of claim 10, wherein at least the portion of the information sent to the one or more medical devices triggers a generation of one or more electronic records documenting an interaction with the one or more medical devices to fulfill the first order.

12. The system of claim 11, wherein the operations further comprise:
receiving a patient encounter command specifying the first patient;
in response to the patient encounter command, retrieving, from an electronic medical record (EMR) repository, one or more orders associated with the first patient;
sending, to the client device, the one or more orders for output at the client device; and
receiving, from the client device, the selection of the first order made based at least on the one or more orders output at the client devices.

13. The system of claim 12, wherein the patient encounter command and the selection of the first order comprise one or more voice inputs, text inputs, and/or haptic inputs received at the client device.

14. The system of claim 13, wherein the scannable token is generated to include a uniform resource locator (URL) of a server from which the one or more medical devices retrieves at least the portion of the information associated with the first order.

15. The system of claim 14, wherein the scannable token is generated to include at least the portion of the information associated with the first order.

16. The system of claim 15, wherein the information associated with the first order is encrypted, and wherein the scannable token is further generated to include a uniform resource locator (URL) of a server from which to retrieve a key for decrypting the information.

17. The system of claim 1, wherein the patient interaction includes a first interaction with a first medical device followed by a second interaction with a second medical device, and wherein an error message is triggered in response to the scannable token being scanned at the second medical device before the first medical device.

18. The system of claim 17, wherein the scannable token is further generated for a second patient encounter to fulfill a second order associated with the first patient or a second patient, and wherein the first patient encounter is prioritized over the second patient encounter based on a scheduled time of each encounter, a first location of the clinician relative to a second location of the first patient and a third location of the second patient, a shift and break scheduling of the clinician, and/or a criticality of each patient.

19. (canceled)

20. The system of claim 1, wherein the operations further comprise:
in response to the scannable token being scanned at the one or more medical devices, sending, to the client device, one or more instructions, reminders, and/or feedback associated with the encounter.

21. A method, comprising:
receiving a selection of a first order associated with a first patient;
generating a scannable token for interacting with one or more medical devices involved in a first patient encounter to fulfill the first order;
sending, to a client device, the scannable token; and
in response to the scannable token being scanned at the one or more medical devices, sending, to the one or more medical devices, at least a portion of information associated with the first order.

22-41. (canceled)
